(19) United States
(12) Patent Application Publication
(10) Pub. No.: US 2025/0345430 A1
(43) Pub. Date: Nov. 13, 2025

(54) PLURIPOTENT STEM CELL-DERIVED IMMUNE CELL INDUCING CHEMOTAXIS FOR HETEROGENEOUS IMMUNE CELLS

(71) Applicant: TSD LIFE SCIENCES CO., LTD., Seoul (KR)

(72) Inventors: Ki Doo CHOI, Suwon-si (KR); Dohyun SON, Namyangju-si (KR); Sanghyeon OH, Seoul (KR); Jeongyi KIM, Seoul (KR); Honggyu AHN, Seoul (KR)

(73) Assignee: TSD LIFE SCIENCES CO., LTD., Seoul (KR)

(21) Appl. No.: 18/860,081

(22) PCT Filed: Apr. 27, 2023
(86) PCT No.: PCT/KR2023/005751

§ 371 Date: Oct. 25, 2024

(30) Foreign Application Priority Data
Apr. 27, 2022 (KR) 10-2022-0051855

(51) Int. Cl. A61K 40/35; A61K 35/17; A61K 40/15; A61P 35/00; C07K 14/52; C07K 14/54; C12N 5/0783

(52) U.S. Cl. CPC A61K 40/35; A61K 35/17; A61K 40/15; A61P 35/00; C07K 14/523; C07K 14/5418; C12N 5/0646

(57) ABSTRACT

A composition for preventing or treating cancer or infectious disease is disclosed. The composition contains, as an active ingredient, a pluripotent stem cell-derived immune cell expressing IL-7, CCL19, or a combination thereof. The composition provides a multifaceted and synergistic therapeutic effect derived from the complementary immune response of the patient's endogenous T cells and the injected natural killer cells, by administering only a therapeutically effective amount of immune cells other than T cells, specifically natural killer cells. The natural killer cells of the composition may also be co-administered with exogenous T cells to allow these different cell populations to act more intensively at the lesion site. The composition is based on the differentiation of pluripotent stem cells, specifically induced pluripotent stem cells (iPSCs) into immune cells, thus can be used to generate an unlimited supply of allogenic or autologous cells as needed.

### TECHNICAL FIELD

The present invention relates to a method for obtaining natural killer cells expressing specific chemotactic factors for migration of heterologous immune cells to a lesion site, using pluripotent stem cells as starting cells.

### BACKGROUND ART

Human pluripotent stem cells (hPSCs), including human embryonic stem cells (hESCs) and induced pluripotent stem cells (iPSCs), can proliferate indefinitely while retaining the ability to differentiate into various types of somatic cells. These cells are valued for their ability to provide an unlimited source for cell therapy and regenerative medicine. Therefore, there are active researches into various methods of inducing differentiation, isolating differentiated cells, and removing undifferentiated cells in order to specifically differentiate them into therapeutic immune cells or cells of the appropriate type for the tissue being regenerated.

Natural killer cells (NK cells) are lymphoid cells that account for about 15% of peripheral blood lymphocytes and play a crucial role in the innate immune response. In particular, they remove tumor cells by activating dendritic cells and inducing cytotoxic T lymphocytes (CTLs) to specifically react to the tumor. Natural killer cells directly kill malignant tumors including sarcomas, myelomas, lymphomas and leukemia, and its therapeutic effect was confirmed by administration of in vitro-activated NK cells to patients with blood cancer, such as leukemia, after bone marrow transplantation (Blood Cells Molecules & Disease, 33: p261-266, 2004). However, since most natural killer cells are in an inactive state in the body of a normal person, studies have been focused on in vitro activation of NK cells isolated from blood for increased treatment efficiency.

On the other hand, T cells, which constitute another axis among immune cells, are lymphocytes that are generated in the bone marrow and matured in the thymus. They have memory functions in the immune system and provide information to B cells to induce antibody production. After undergoing an immune tolerance test in the thymus, T cells differentiate into 4 types: cytotoxic T cells, helper T cells, regulatory T cells and memory T cells. Cytotoxic T cells, which are CD8+, bind to type I MHC to identify cancer cells or infected cells, and mainly target lesional cells like natural killer cells.

Since therapeutic effects can be maximized when natural killer cells and cytotoxic T cells are administered in combination, the development of novel therapies that overcome the inefficiency of injecting two cells with each therapeutically effective amount is required.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

The present inventors have made intensive studies to develop efficient cellular therapeutic compositions that eliminate diseased cells or tissues more efficiently through a complex immune response by a combination of heterologous immune cells, while being readily available in therapeutically effective amounts. As a result, the present inventors found that when pluripotent stem cells are transfected with IL-7 gene, CCL19 gene or a combination thereof, and then differentiated into immune cells, more specifically, immune cells other than T cells, most specifically, natural killer cells, in vivo injection of these differentiated natural killer cells may induce a multifaceted and synergistic immune response by simultaneously acting on the lesion with endogenous T cells proliferated and homed by IL-7 and CCL19 expressed by the natural killer cells.

Accordingly, it is an object of this invention to provide a pluripotent stem cell-derived immune cells expressing IL-7, CCL19, or combinations thereof; methods of preparing the same, and compositions for the preventing or treating cancer or infectious diseases comprising the same as active ingredients.

Other objects and advantages of the present invention will become more apparent from the following detailed description, the appended claims, and the accompanying drawings.

### Technical Solution

In one aspect of this invention, there is provided an immune cell that is differentiated from a pluripotent stem cell and expresses a nucleic acid molecule encoding IL-7 (interleukin-7) or a functional portion thereof; a nucleic acid molecule encoding CCL19 (C-C Motif Chemokine Ligand 19) or a functional portion thereof; or a combination thereof.

The present inventors have made intensive studies to develop efficient cellular therapeutic compositions that eliminate diseased cells or tissues more efficiently through a complex immune response by a combination of heterologous immune cells, while being readily available in therapeutically effective amounts. As a result, the present inventors found that when pluripotent stem cells are transfected with IL-7 gene, CCL19 gene or a combination thereof, and then differentiated into immune cells, more specifically, immune cells other than T cells, most specifically, natural killer cells, in vivo injection of these differentiated natural killer cells may induce a multifaceted and synergistic immune response by simultaneously acting on the lesion with endogenous T cells proliferated and homed by IL-7 and CCL19 expressed by the natural killer cells.

The term “immune cell” as used herein refers to any cell involved in the initiation or promotion of an immune response, and more particularly, an immune effector cells. Immune cells include, for example, T cells, B cells, natural killer (NK) cells, natural killer T (NKT) cells, mast cells and dendritic cells, but are not limited thereto. More specifically, the immune cell is an immune cell other than T cell, and most specifically, a natural killer cell.

The term “T cell” as used herein refers to a group of cells in the same scope as those understood in the art and includes CD8+ or CD4+ T cells that directly lyses target cells or provides an effector function or helper function which causes the death of target cells, but is not limited thereto and may include any cell classified as “T cell” in the art to which the present invention pertains.

The term “stem cell” as used herein refers to an undifferentiated cell in the stage prior to differentiation into each of the cells that make up a tissue, thus is a collective term for cells that have the ability to differentiate into specific cells in the presence of a specific differentiation stimulus or environment. Unlike differentiated cells, which have stopped dividing, stem cells are characterized by their ability of self-renewal by cell division. When a differentiation stimulus is applied, stem cells may differentiate into various cells depending on the nature of the stimulus, which is called differentiation plasticity.

The stem cells may be used in the present invention without limitation as long as they have the characteristics of stem cells, i.e., undifferentiated, indefinitely proliferating, and capable of inducing differentiation into the tissue to be regenerated.

The term “pluripotent stem cell” as used herein refers to a stem cell that is at a more advanced developmental stage than a fertilized egg and is capable of differentiating into any cell constituting endoderm, mesoderm, and ectoderm. According to specific embodiments, the pluripotent stem cells used herein are Embryonic Stem Cells (ESCs), Embryonic Germ Cells, Embryonic Carcinoma Cells, or induced Pluripotent Stem Cells (iPSCs), more specifically Embryonic Stem Cells or induced Pluripotent Stem Cells, and most specifically induced Pluripotent Stem Cells.

The term “induced pluripotent stem cell” as used herein refers to a type of pluripotent stem cell artificially derived by inserting specific genes associated with an undifferentiated or pluripotent phenotype into a non-potent cell (e.g., somatic cell). Induced pluripotent stem cells are considered in the art to have the same phenotypic, physiological, and embryological characteristics as naturally occurring pluripotent stem cells, such as embryonic stem cells, including expression of stem cell gene and protein, chromosomal methylation, doubling time, germ body formation, teratoma formation, viable chimerism, hybridization, and differentiation.

The term “differentiation of a stem cell” as used herein includes not only the induction of complete differentiation of an undifferentiated stem cell into a specific cell, but also the formation of precursor cells that form at an intermediate stage prior to complete differentiation of a stem cell into a specific cell.

The term “functional portion” as used herein refers to an equivalent fragment of full-length protein form where certain amino acid residues are deleted, which maintains original biological activity and function of the full-length protein.

The term “nucleic acid molecule” as used herein has comprehensive meaning including DNA (gDNA and cDNA) and RNA molecule. A nucleotide, which is a basic construct unit of nucleic acid molecule, includes nucleotide analogues with modified sugar or base, as well as natural-occurring nucleotides (Scheit, Nucleotide Analogs, John Wiley, New York (1980); Uhlman and Peyman, Chemical Reviews, 90:543-584 (1990)).

It would be obvious to the skilled artisan that the nucleotide sequences used in this invention are not limited to those listed in the appended Sequence Listings. For nucleotides, the variations may be purely genetic, i.e., ones that do not result in changes in protein structure due to codon degeneracy or the presence of different codons coding for biologically equivalent amino acids. Considering these biologically equivalent variations, the nucleic acid molecule of this invention may encompass sequences having substantial identity to them. Substantial identity means a sequence that exhibits at least 80% homology, more specifically 85% homology, more specifically 90% homology, more specifically 90% homology, and most specifically 95% homology when the sequence of the present invention is aligned with any other sequence as closely as possible and the aligned sequence is analyzed using an algorithm known in the art.

The term “express” as used herein includes artificially expressing an exogenous gene that is not naturally expressed by the immune cell of the present invention through a gene carrier, naturally expressing an endogenous gene through an endogenous expression system, or overexpressing endogenous using a gene carrier to increase the amount of natural expression of the endogenous gene. Therefore, the immune cell of the present invention includes a cell endogenously expressing IL-7 and CCL19, a cell endogenously expressing IL-7 and artificially expressing CCL19, a cell endogenously expressing CCL19 and artificially expressing IL-7, and a cell artificially expressing IL-7 and CCL19.

The term “to express” as used herein refers to being artificially replicated as an extrachromosomal factor or by chromosomal integration in a target cell via a gene delivery system to cause the target cell to express a foreign gene or overexpress an endogenous gene. Accordingly, “express” in the term “to express” may be used interchangeably with “transformation”, “transfection”, or “transduction”.

The “gene delivery system” as used herein refers to any means of delivering a gene into a cell. The gene delivery has the same meaning as intracellular transduction of genes. At the tissue level, the term gene delivery has the same meaning as the spread of a gene. Accordingly, the gene delivery system of the present invention can be described as a gene penetration system or a gene spread system.

The nucleotide sequences of the IL-7 and CCL19 gene may be applied to all gene delivery systems used for conventional gene transfers, such as plasmid, adenovirus, adeno-associated virus, retrovirus, lentivirus, herpes simplex virus, vaccinia virus, liposom or niosome.

When the gene delivery system of the present invention is a naked recombinant DNA molecule or a plasmid, the gene can be introduced into cells by microinjection (Capecchi, M. R., Cell, 22:479 (1980)), calcium phosphate precipitation (Graham, F. L. et al., Virology, 52:456 (1973)), electroporation (Tur-Kaspa et al., Mol. Cell Biol., 6:716-718 (1986)), liposomemediated transfection (Biochim. Biophys. Acta, 721:185-190 (1982)), DEAE-dextran treatment (Gopal, Mol. Cell Biol., 5:1188-1190 (1985)) and gene bombardment (Yang et al., Proc. Natl. Acad. Sci., 87:9568-9572 (1990)), and more concretely, liposome-mediated transfection methods can be used to introduce the gene into the cell.

The IL-7 and CCL19 genes may be inserted together in one gene delivery system or separately in two gene delivery system included in the composition.

According to a concrete embodiment, the IL-7 comprises the amino acid sequences with at least 85% sequence similarity, more concretely at least 90% similarity, and most concretely at least 95% sequence similarity with the amino acid sequence of SEQ ID NO:1.

According to a concrete embodiment, the CCL19 comprises amino acid sequences with at least 85% or more similarity, more concretely at least 90% or more sequence similarity, and most concretely at least 95% or more sequence similarity with the amino acid sequence of SEQ ID NO:2.

According to a concrete embodiment, nucleic acid molecules encoding IL-7 or a functional portion thereof comprises the nucleotide sequence of SEQ ID NO:3.

According to a concrete embodiment of the present invention, nucleic acid molecules encoding CCL19 or a functional portion thereof comprises the nucleotide sequence of SEQ ID NO:4.

According to the present invention, SEQ ID NO:3 and SEQ ID NO:4 are codon optimized nucleotide sequences for IL-7 and CCL19, respectively, for effective expression in natural killer cells.

To artificially express IL-7 and CCL19 in the immune cells of the present invention, pluripotent stem cells can be transfected with these genes and then induced to differentiate into immune cells, or pluripotent stem cells can be differentiated into immune cells and then transfected with these genes at the stage of differentiated immune cells.

According to a specific embodiment of the present invention, the pluripotent stem cells are transfected with a nucleic acid molecule encoding IL-7 or a functional portion thereof; a nucleic acid molecule encoding CCL19 or a functional portion thereof.

In another aspect of this invention, there is provided a composition for preventing or treating cancer or an infectious disease comprising the immune cell of the present invention described above as an active ingredient.

In still another aspect of this invention, there is provided a method for preventing or treating cancer or an infectious disease comprising administering the immune cell of the present invention described above to a subject in need thereof.

In case the immune cells of the present invention are applied as cell therapy, it may be utilized for the treatment of various tumors and infectious diseases. The immune cells of the present invention, in particular natural killer cells, can be applied to all types of tumors including solid cancer and blood cancer. Such cancer may include, but not limited to, gastric cancer, liver cancer, lung cancer, colorectal cancer, breast cancer, prostate cancer, ovarian cancer, pancreatic cancer, cervical cancer, thyroid cancer, laryngeal cancer, acute myelogenous leukemia, brain tumor, neuroblastoma, retinoblastoma, head and neck cancer, salivary gland cancer and lymphoma. Infectious diseases that may be prevented or treated by the immune cells of the present invention, particularly natural killer cells, may be diseases caused by viral or pathogenic infections, which include all diseases that can be infected through respiratory, blood, skin contact, and the like. Such infectious disease include, but are not limited to, hepatitis B and C, human papilloma virus (HPV) infection, cytomegalovirus infection, viral respiratory disease and influenza.

The term “preventing” as used herein refers to inhibiting the occurrence of a disorder or disease in a subject who has never been diagnosed as having the disorder or disease, but is at risk of developing the condition or disease.

The term “treating” as used herein refers to (a) inhibiting the progress of a disorder, disease or symptom; (b) alleviating the disorder, disease or symptom; or (c) eliminating the disorder, disease or symptom. When the immune cells of the present invention are administered to a subject, a complex immune response is generated by natural killer cells and endogenous T cells or exogenously injected autologous or allogeneic T cells recruited to the lesion site by the natural killer cells, which serves to inhibit, eliminate or alleviate the development of symptoms caused by tumors or infectious diseases by inducing the killing of cancer cells, infected cells, or pathogens. Therefore, the composition of the present invention may itself be a cell therapy composition, or may be applied as a therapeutic adjuvant for the disease by being administered together with other active ingredients, such as therapeutic T cells or other known anticancer agents. Accordingly, the term “treatment” or “therapeutic agent” in the present specification includes the meaning of “therapeutic aid” or “therapeutic adjuvant”.

The term “administration” or “to administer” as used herein refers to the direct administration of a therapeutically effective amount of the composition of the present invention to a subject so that an equal amount is formed in the body of the subject.

The terms “therapeutically effective amount” as used herein refer to the content of the composition of the present invention that is sufficient to provide a therapeutic or prophylactic effect to a subject to whom the composition is to be administered, and thus include the meaning of a “prophylactically effective amount”.

The term “subject” as used herein includes, without limitation, humans, mice, rats, guinea pigs, dogs, cats, horses, cows, pigs, monkeys, chimpanzees, baboons or rhesus monkeys. Concretely, the subject of the present invention is humans.

In still another aspect of this invention, there is provided a composition for inducing proliferation or homing of a heterogeneous immune cell comprising an immune cell of the present invention as an active ingredient.

In still another aspect of this invention, there is provided a method for inducing proliferation or homing of a heterogeneous immune cell comprising administering to a subject an immune cell of the present invention as described above.

The nucleic acid molecules used in the present invention and the pluripotent stem cell-derived immune cells into which the nucleic acid molecules are transfected have already been described above in detail and are therefore omitted to avoid undue redundancy.

The term “heterogeneous immune cells” used herein refers to a type of immune cell that is different from the immune cells of the present invention that are differentiated from stem cells. Concretely, the heterogeneous immune cells are T cells or dendritic cells. According to the present invention, a large amount of endogenous T cells can be migrated to the lesion by injecting immune cells simultaneously expressing IL-7 and CCL19, concretely immune cells other than T cells, and most concretely natural killer cells, into the subject to induce proliferation and chemotaxis of T cells. The term “chemotaxis” refers to the positive (towards a stimulus) or negative (away from a stimulus) migration of cells which is induced by chemical stimuli. More concretely, the term “chemotaxis” refer to positive migration. According to the present invention, T cells are proliferated and differentiated by IL-7 and activated by CCL19, a corresponding chemotactic factor, and then move toward natural killer cells expressing IL-7 and CCL19. This results in formation of heterogeneous cell population composed of two types of complementary immune cells, i.e. T cells and natural killer cells, around the lesion site. Accordingly, the present invention provides the effect of co-administration of different cell populations by injecting only a therapeutically effective amount of a single cell type, namely natural killer cells.

In still another aspect of this invention, there is provided a method for preparing a transformed immune cell comprising:
(a) introducing into a pluripotent stem cell a nucleic acid molecule encoding interleukin-7 (IL-7) or a functional portion thereof; a nucleic acid molecule encoding C-C Motif Chemokine Ligand 19 (CCL19) or a functional portion thereof; or a combination thereof; and
(b) differentiating the cells obtained in the step (a) into an immune cell.

The nucleic acid molecules used in the present invention, the pluripotent stem cells into which they are introduced, and the immune cells differentiated therefrom have already been described above in detail and are therefore omitted to avoid undue redundancy.

In still another aspect of this invention, there is provided a pluripotent stem cell expressing a nucleic acid molecule encoding IL-7 (interleukin-7) or a functional portion thereof; a nucleic acid molecule encoding CCL19 (C-C Motif Chemokine Ligand 19) or a functional portion thereof; or a combination thereof.

The nucleic acid molecules used in the present invention and the pluripotent stem cells into which they are introduced have already been described above in detail and are therefore omitted to avoid undue redundancy.

The IL-7 and/or CCL19-expressing pluripotent stem cells of the present invention, specifically induced pluripotent stem cells (iPSCs), can serve as efficient immunotherapeutic cells with immunopotentiating activity by themselves, as they can induce proliferation and homing to lesions of endogenous T cells or exogenously injected T cells, as well as easily reach therapeutically effective amounts through infinite proliferation. In addition, they can be used as starting cells for obtaining target cells expressing IL-7 and/or CCL19 by appropriate differentiation stimulation, thereby producing large quantities of various adult immune cells, mesenchymal stem cells, and other cells for tissue regeneration that can interact synergistically with T cells.

### Advantageous Effects

The features and advantages of the present invention are summarized as follows:
(a) The present invention provides a composition for preventing or treating cancer or infectious disease comprising, as an active ingredient, a pluripotent stem cell-derived immune cell expressing IL-7, CCL19, or a combination thereof.
(b) The present invention provides a multifaceted and synergistic therapeutic effect derived from the complementary immune response of the patient's endogenous T cells and the injected natural killer cells, by administering only a therapeutically effective amount of immune cells other than T cells, specifically natural killer cells.
(c) The natural killer cells of the present invention may also be co-administered with exogenous T cells to allow these different cell populations to act more intensively at the lesion site.
(d) The present invention is based on the differentiation of pluripotent stem cells, specifically induced pluripotent stem cells (iPSCs) into immune cells, thus can be used to generate an unlimited supply of allogenic or autologous cells as needed. Therefore, the present invention may be applied to obtain large quantities of highly scarce immunotherapeutic cell resources through infinite proliferation.

### BRIEF DESCRIPTION OF DRAWINGS

FIG. 1 represents a schematic diagram showing the structure of the pEF1-IRES empty vector used as a negative control vector.

FIG. 2 represents a schematic diagram showing the structure of the pEF1-IRES vector into which IL-7 and CCL19 genes are inserted.

FIG. 3 represents a schematic diagram showing the structure of the pEF1-IRES vector into which CCL19 and IL-7 genes are inserted.

FIG. 4 is a schematic illustrating for the process of differentiating NK cells from iPS cells.

FIG. 5 shows the results of measuring the expression of CCL19 and IL-7 in NK cells (7×19 iNK) differentiated from iPS cells with inserted CCL19 and IL-7 genes.

FIG. 6a shows the results of producing embryoid bodies (EBs) by spin-EB method from the iPS cell state (D+0), differentiating them (D+1), and then inducing their differentiation into hematopoietic stem cells (HSCs) for 6 days. FIG. 6b shows that 39.64% CD3-CD56+ cells were obtained after 28 days of induced differentiation from hematopoietic stem cells to NK cells (D+34). FIG. 6c shows the differentiation of 63.81% CD3-CD56+ cells by further 7 days of differentiation induction (D+41).

FIG. 7 illustrates the anti-cancer activity of NK cells differentiated from iPS cells as measured by LDH assay.

FIG. 8 shows the results of measuring CD107a expression in NK cells differentiated from iPS cells.

FIG. 9 illustrates the results of comparing the antitumor effects of 7×19 iNK and iNK cells without gene transduction. FIG. 9a shows the results of cytotoxicity measurements on the HepG2 cell line, and FIG. 9b represents FACS graphs showing the induction of apoptosis in the HepG2 cell line.

FIG. 10 represents the effect of T cell migration by 7×19 iNK cells, showing the fold change of migrated T cells (FIG. 10a), number of migrated cells (FIG. 10b), and the image for migrated T cells for each culture condition (FIG. 10c).

### MODE FOR INVENTION

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### EXAMPLES

### Example 1

### Construction of IL-7 and CCL19 Expression Vectors

An expression vector was constructed to produce human NK cells expressing IL-7 and CCL19. pEF1a-IRES bicistronic mammalian expression vector (Takara, CAT #631970) was used as the expression vector under the control of the human elongation factor 1 alpha (EF1α) promoter.

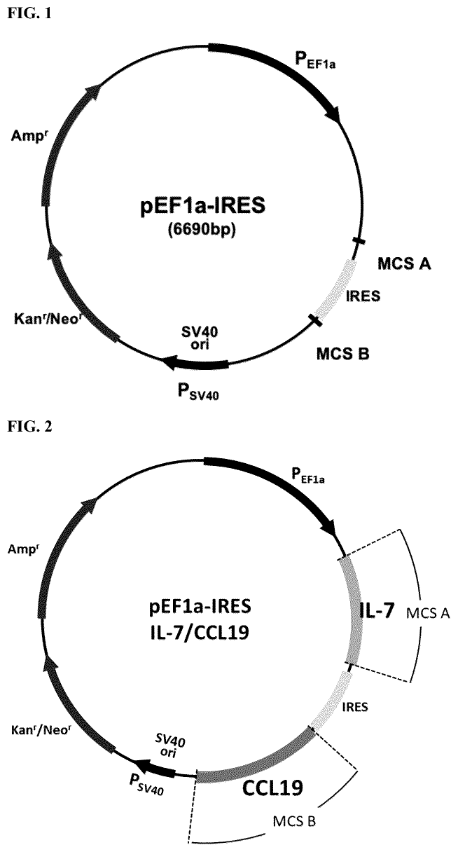

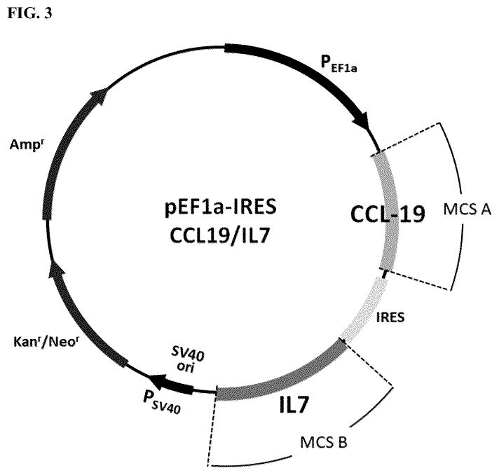

The ORF sequences of IL-7 and/or CCL19, the genes to be inserted into the vector, were synthesized by codon optimization (SEQ ID NOs: 3 and 4, respectively), and IL-7 or CCL19 was cloned into multicloning site A (MCS A) of the pEF1a-IRES vector. CCL19 or IL-7 was cloned into MCS B. The internal ribosome entry site (IRES) was placed between the two MCSs to allow co-expression of the two genes. The pEF1a-IRES vector has an internal ribosome entry site (IRES) located between the two MCSs to allow the two genes to be co-expressed. The structure of empty vector used as negative control is shown in FIG. 1, the structure of the IL-7/CCL19 expression vector is shown in FIG. 2, and the structure of the CCL19/IL7 expression vector is shown in FIG. 3.

### Example 2

### Preparing IL-7 and CCL19-Expressing iPS Cell and Verification of Expression

The iPS cell line purchased from Thermofisher (CAT #A18945) were coated in 75T flasks by diluting Matrigel (CorningR, CAT #354277) in DMEM/F12 (1:100) and cultured in medium containing mTeSR™ Plus (CAT #100-0276) and 10 uM Y-27632.

To generate IL-7/CCL19 expressing iPS cell lines (hereinafter, ‘7×19 iPS cell line’), 2 mL/well were seeded into 6-well plates at a density of 1×10⁶ cells to generate transduced 7×19 iPS cell lines 24 hours later.

The 7×19 expression vector was transfected into iPS cells using Lipofectamine™ 3000 (Invitrogen, CAT #L3000015). In brief, 1 μg DNA (7×19 expression vector or negative control vector made in Example 1) was transfected with 5 μL of Lipofectamine™ 3000 followed by 5 min incubation, and then the cells transfected with 7×19 iPSC or negative control (empty-vector) were screened by treating with 50 g/mL G418 (Invitrogen, CAT #04727878001).

To obtain cell lines with stable 7×19 expression as single cell clones, the cells were cultured in medium containing 50 mg/mL G418 for 4 weeks, during which time each single cell from the wells that secreted 7×19 was transferred to a new 6-well plate. Multiple passages were performed to generate single cell-derived clones, thereby the clones consistently secreting 7×19 were selected to obtain 7×19 iPS stable cell lines. To confirm the expression of 7×19 gene, the cell lines were cultured for 24 hours to measure the expression of IL-7 and CCL19. The culture medium was collected in a 6-well plate and the supernatant was separated by centrifugation at 800 g for 5 minutes, and 100 mL per well of the supernatant was used for IL-7 or CCL19 enzyme linked immunosorbent assay (ELISA; IL-7, Komabiotech, CAT #k0331215; CCL19, Abcam, CAT #ab100601). Immunosorbent assay experiments for IL-7 and CCL19 were performed according to the standard methods of each manufacturer, and the final values measured at a wavelength of 450 nm are shown in Table 1.

TABLE 1

| | IL-7 secretion | CCL19 secretion |
|---|---|---|
| Experimental group | (pg/mL) | (pg/mL) |
| Negative control cells | <Min | <Min |
| 7 × 19 iPS | 3232.0 | 425.39 |
| stable cell line | | |

The results in Table 1 show that the transfected 7×19 iPS cell line secreted 3232.0 pg/mL and 425.39 pg/mL of IL-7 and CCL19, respectively. In contrast, IL-7 and CCL19 were undetectable in the negative control cell line transfected with the empty-vector.

### Example 3

### Anti-Cancer Activity of iPS Cell-Derived NK Cells

To measure the anticancer activity of iPS cell-derived NK cells and 7×19 NK cells, LDH cytotoxicity assay (Promega, CAT #G1780) was performed. As a control, NK cells isolated from donor PBMCs (CD3−, CD56+) were cultured in NK MACS (Miltenyi Biotec, CAT #130-114-429) containing 5% human serum (Sigma-Aldrich CAT #H4522) for 21 days. PBNKs and the liver cancer cell line HepG2 (ATCC CAT #HB8065) were then co-cultured in RPMI 1640 (CAT #A1049101) containing 10% FBS (Gibco CAT #10099141) to perform LDH cytotoxicity assays.

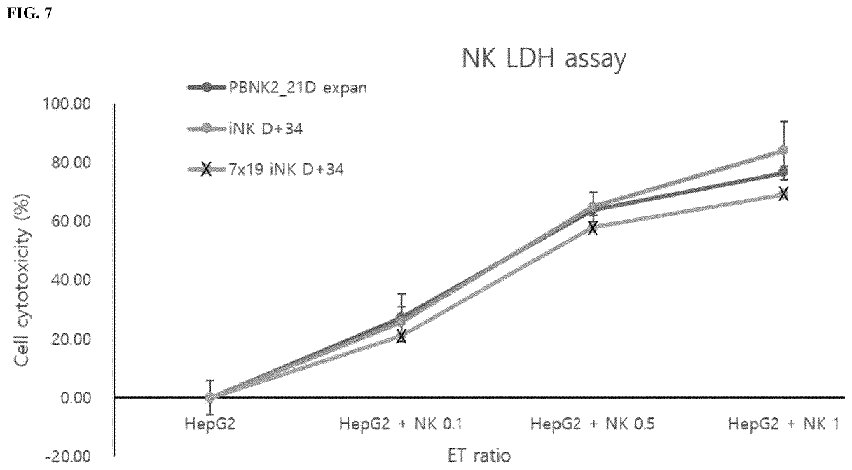

Target cells (HepG2) cultured for one day were treated with effector cells (iPSC-derived NK, 7×19 iPSC-derived NK, PBMC-derived NK) and incubated in a 37° C. incubator for 4 hours. To measure the Max LDH value, the target cells were treated with lysis buffer (9% Triton X-100) as a positive control, and the LDH in the culture medium itself was measured as a negative control. The results were subjected into Equation 1 below and the cytotoxicity according to effector: target cells ratio is shown in FIG. 7. As seen in FIG. 7, the cytotoxicity increased as the ratio of iNK cells increased, and iNK cells exhibited higher cytotoxicity than PBMC-derived NK cells.

$$Experimental = E / T cell ( avg . ) LDH release - Culture medium background ( avg . ) \qquad ( 1 )$$

$$Target Spontaneous = Target cell spontaneous LDH release ( avg . ) - Culture medium background ( avg . ) \qquad ( 2 )$$

$$Effecter Spontaneous = Effecter cell spontaneous LHD release ( avg . ) - Culture medium background ( avg . ) \qquad ( 3 )$$

$$Target Maximum = Target cell maximum LDH release ( avg . ) - Volume correction control ( avg . ) \qquad ( 4 )$$

$$[ Equation 1 ]$$

$$Cytotoxicity ( \% ) = \frac{Experimental - EffecterSpontaneous - TargetSpontaneous}{TargetMaximum - TargetSpontaneous} \times 100$$

$$= ( 1 - 2 - 3 ) / ( 4 - 2 )$$

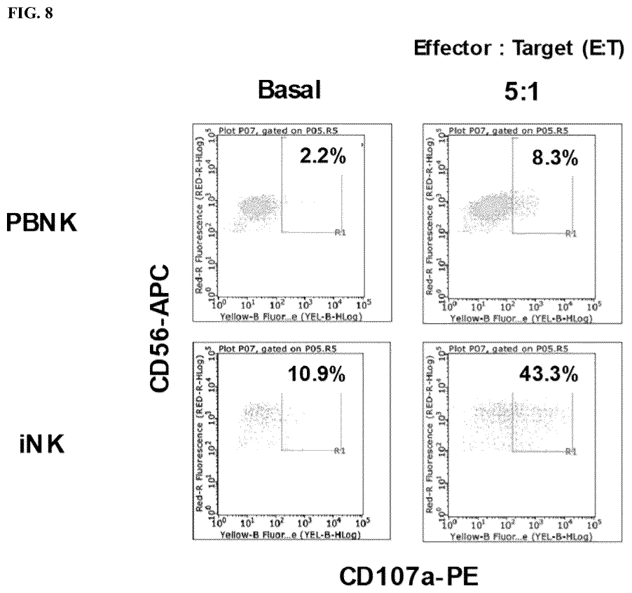

To further evaluate the anti-cancer activity of iPS cell-derived NK cells, a CD107a degranulation assay was performed. CD107a is a marker of NK cell functional activity that is upregulated on the surface of NK cells stimulated by MHC-deficient targets appearing on a subset of tumor cells. For detecting CD107a expressed on NK cells, the expression of CD107a was measured using an anti-CD107a-PE antibody (Invitrogen, CAT #12-1079-42) after co-culture of target cells, K562 (ATCC, CAT #CCL-243), with effector cells, peripheral blood-derived NK cells (PBNK, donor blood) and iPSC-derived NK cells (INK, made in-house) at a 1:5 ratio. Experimental and control groups were treated with CD107a-PE antibody for 1 hour and then reacted with BD Golgistop (BD Bioscience, CAT #554724), a protein transport inhibitor, for 4 hours after 1× treatment to prevent the loss of stained CD107a. Among the effector cells co-cultured with target cells, 43.4% of the CD3−/CD56+ cells showed increased expression of CD107a, which was more than 4-fold higher than the control group (10.9%) and even higher than PBNK-derived NK cells (8.3%) (FIG. 8).

### Example 4

### Differentiation of iPS Cells Into Immune Cells

For inducing the differentiation of the iPS cells into immune cells, the processes involving differentiation into hematopoietic stem cells (HSCs) (step 1) and differentiation into immune cells (step 2) are applied. For step 1, iPS cell lines cultured in 6-well plates or 25T flasks were transferred to 96-well plates and differentiated by spin-EB (300 g, 5 min) with HSC differentiation medium (STEMdiff AFEL2, STEMCELL technology CAT #05275) containing Y-27632 along with BMP-4 (R&D systems CAT #314-BP-050), VEGF (R&D systems CAT #293-VE-050), SCF (Peprotech CAT #300-07). Cells were cultured for 6 days while observing the morphology of EBs (Embryoid Bodies). HSC differentiation was confirmed by CD34 expression, a representative marker of HSCs. As a result, 66% of the cells were differentiated into HSCs.

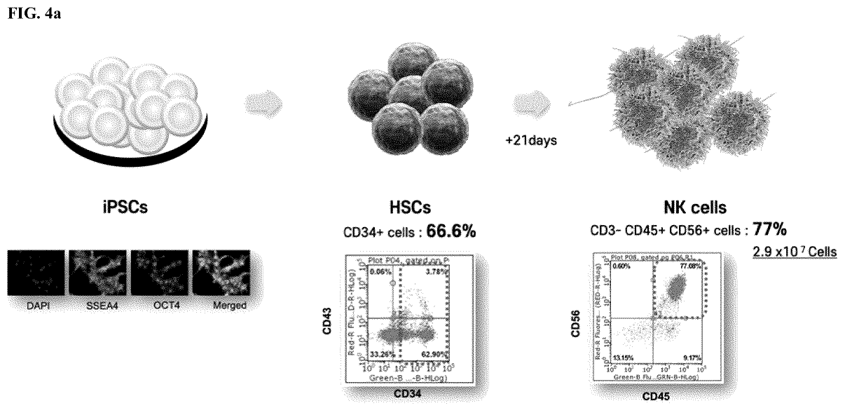

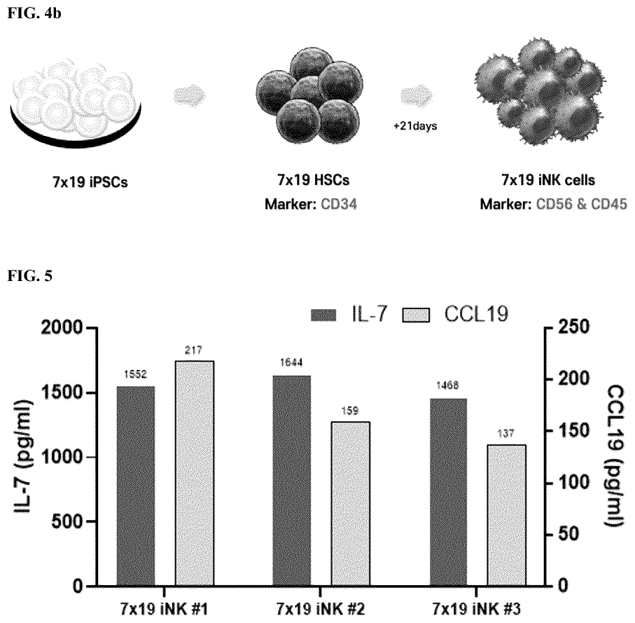

For the second stage of differentiation to obtain NK cell, the HSC differentiation medium was removed and replaced with NK cell differentiation medium containing IL-3 (Peprotech CAT #200-3), IL-7 (Peprotech CAT #200-07), IL-15 (Peprotech CAT #200-15), SCF and FLT3L (Peprotch CAT #300-19) and cultured for 6 days. After 21 days of culture, NK cells co-expressing CD56 and CD45 while not expressing CD3 were selected by FACS, and iPS cell line-derived NK cells (iNK) were obtained at a rate of 77% (FIG. 4a).

### Example 5

### Differentiation of IL-7 and CCL19-Expressing Ips Cells Into Immune Cells

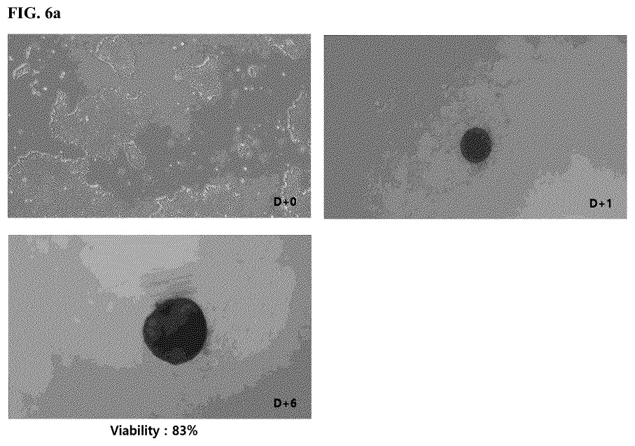

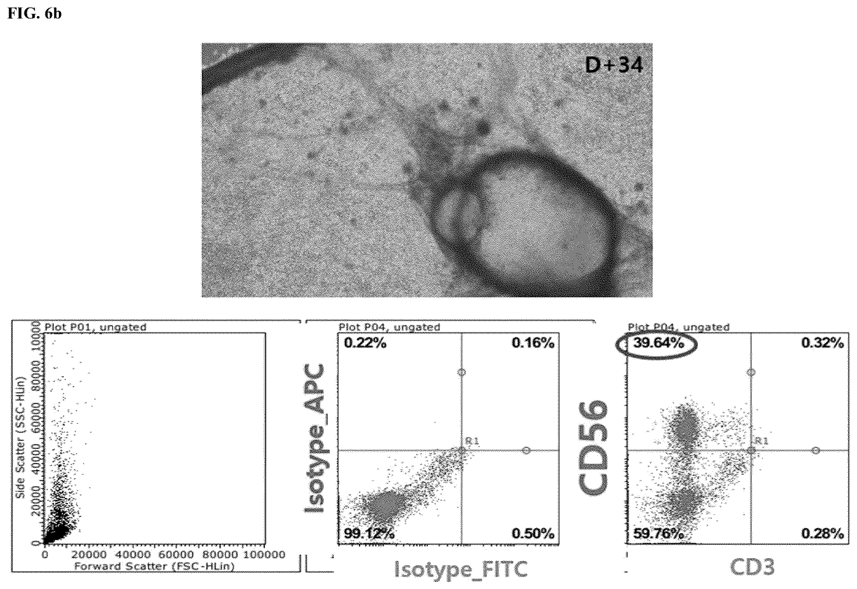

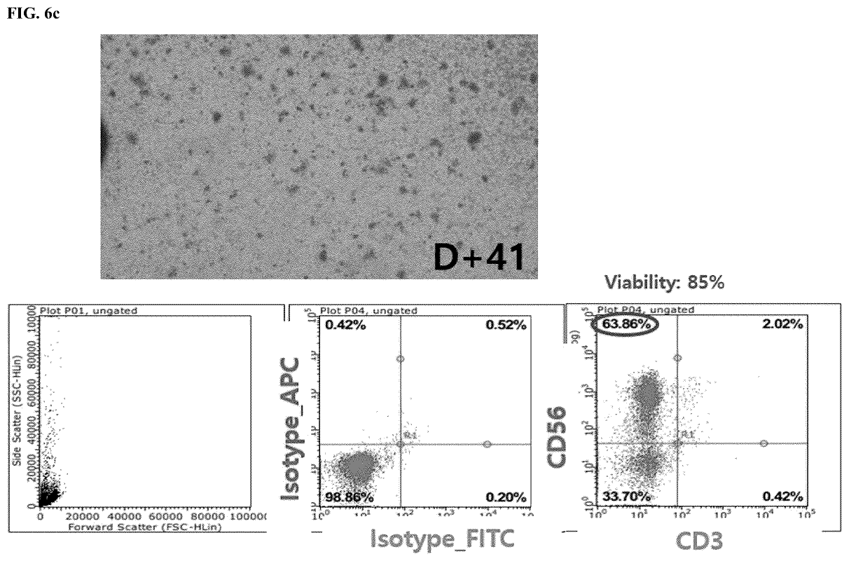

Using the 7×19 iPS generated in Example 2 as a starting cell, IL-7 and CCL19 expressing NK cells were prepared by the same process as described in Example 4 (FIG. 4b). Six clones of RCBs were established by selecting monoclonal 7×19 iPS, and the three 7×19 iPSCs with the most stable dual expression were differentiated into NK cells (7×19 iPSC-dervied NK cells, 7×19 iNK) by the method of Example 4. The analysis for the expression of IL-7 and CCL19 in the cultures of 7×19 iNKs revealed that all cells stably secreted IL-7 and CCL19 (FIG. 5). Each cell showed 83% viability at day 6 of differentiation (FIG. 6a). By inducing differentiation of hematopoietic stem cells which were differentiated from embryoid bodies (EBs), 70% of CD3-CD56+ NK cells were ultimately obtained.

### Example 6

### T Cell Chemotaxis and Proliferation by IL-7 and CCL19 Expressing NK Cells

To determine the effect of IL-7 and CCL19 secreted by NK cells on T cell chemotaxis and proliferation, 12-well transwell (Corning, CAT #CLS3421) with the chambers filtered with 5 μm polycarbonate was used.

### Comparison of IL-7×CCL19 NK92 Cell With and Negative Control Using HuT78 T Cell Line

1×10⁷ of HuT78 cells (ATCC, CAT #TIB-161) were cultured in IMDM (Gibco, CAT #12440053) medium without FBS and supplemented with 1% PS for 24 hours. Then, 10 μl of HuT78 cells at a density of 5×10⁶ cells/mL were seeded into the upper chamber using the same medium as above, and the lower layer of the chamber was seeded with cultures from the IL-7/CCL19 NK92 cell line prepared in Example 2 for 2-3 days.

Then, after 1 day of incubation in 37° C., 5% CO₂ incubator, the upper chamber was removed and incubated for another 3 days. To measure the number of cells in the lower chamber, 400 μl of medium per well was centrifuged at 800 g for 5 min to remove the supernatant to obtain a concentrated sample. 10 μl of the concentrated sample was mixed with 10 μl of Trypan blue in a 1:1 ratio and measured by Countess™ II (Invitrogen, CAT #AMQAX1000). The results were converted to the total number of viable cells in 400 μl and shown in Table 2 below.

TABLE 2
Chemotaxis of HuT78 cell line induced by transgenic NK cells

| | Number of cells in |
|---|---|
| | the lower chamber |
| Experimental group | (mean ± standard deviation) |
| Negative control cells | 3,836 ± 276 |
| IL-7 × CCL19 NK92 cells | 11,989 ± 887 |

### Comparison of IL-7×CCL19 NK92, CCL19×IL-7 NK92 and Negative Controls Using PBMC-Derived T Cells

To determine the chemotaxis of T cells isolated from peripheral blood mononuclear cells (PBMCs), T cells were isolated from PBMCs using the EasySep™ Human T cell Isolation Kit (STEMCELL #17951). T cells at a concentration of 2×10⁶ cells/mL were diluted in RPM11640 medium containing 2% FBS, and 100 μl were dispensed into the upper chamber. The lower chamber was filled with cultures from IL-7×CCL19 and CCL19×IL-7 NK92 cells prepared in Example 2 for 1 day.

After 1 day of incubation in 37° C., 5% CO₂ incubator, the upper chamber was removed and incubated for another 3 days. To measure the number of cells in the lower chamber, 500 μl of medium per well was centrifuged at 800 g for 5 min to remove the supernatant to obtain a concentrated sample. 10 μl of the concentrated sample was mixed with 10 μl of Trypan blue in a 1:1 ratio and measured with Countess™ II. The results were converted to total cell counts and shown in Table 3 below.

TABLE 3
Chemotaxis of PBMC-derived T-cell anergy
induced by a transgenic NK92 cell

| | | Number of T cells that |
|---|---|---|
| | | migrated to the lower |
| | | chamber (mean ± |
| | Experimental group | standard deviation, |
| T cell types | (n = 2) | 10⁴ cells) |
| PBMC-derived T | Negative control | 1.89 ± 0.75 |
| cells | NK92 cells | |
| | IL-7 × CCL19 | 9.35 ± 0.53 |
| | NK92 cells | |
| | CCL19 × IL-7 | 6.46 0.16 |
| | NK92 cells | |

As shown in Tables 2 and 3, the number of T cells migrated to the lower chamber was significantly higher in the group treated with IL-7/CCL19 NK92 and CCL19/IL-7 NK92 cell line compared to the negative control treated with NK92 cell line transduced with empty vector. This confirmed the induction of chemotaxis of T cells and proliferation of migrated T cells by NK92 cell line transduced to secrete IL-7 and CCL19.

### Example 7

The Cancer Cell Killing Effect of a Transgenic NK92 cells and Migrated T Cells, and the Secreted Factors Associated Therewith

The HepG2 liver cancer cell line (ATCC #HB-8065) was diluted in RPMI1640 medium containing 10% FBS at a concentration of 2×10⁵ cells/mL and 0.1 mL was dispensed into each well of a 96-well plate and cultured for 1 day. 0.5 mL RPMI1640 medium containing the NK92 cell line transformed by electroporation was transferred to the lower layer of the transwell chamber in the same way as in Example 6, and 2×10⁵ cells/0.1 mL of T cells isolated from PBMCs were added to the upper chamber to induce T cell migration for 1 day. 1×10⁵ CD3/CD28 Dynabeads and 100 U of IL-2 were added to the lower chamber to activate the migrated T cells. After 1 day of incubation, the upper chamber was removed and 0.1 mL per well was transferred to a 96-well plate where HepG2 liver cancer cell lines were cultured and incubated for another 3 days. The plates were then washed three times with Phosphate Buffer Saline (PBS) and replaced with 0.1 mL of 10% FBS-RPMI1640 medium containing 1% CCK-8 solution and then incubated for another 2 hours. To measure the cell viability of the HepG2 liver cancer cell line, the absorbance was measured at 450 nm and the cell viability was calculated as shown in [Equation 2]. The results are shown in Table 4.

$$[ Equation 2 ]$$

$$HepG 2 cell line viability = 100 \times \frac{Absorbance of Experimental group - Absorbance of RPMI 1640 medium}{Absorbance of Control group - Absorbance of RPMI 1640 medium}$$

In addition, the secretion of granzyme-B (Grz-B), interferon-γ (IFN-γ), and tumor necrosis factor (TNF)-α from activated T cells and activated NK cells was measured by enzyme-linked immunosorbent assay (ELISA; GrzB, abcam #ab235635; IFN-γ, komabiotech #K0331121; TNF-α, komabiotech #K0331131), and the results are shown in Table 5. Each value in Table 4 and Table 5 is expressed as mean±standard deviation.

TABLE 4
Killing effect for HepG2 liver cancer cell line

| Cancer cell lines | Experimental group (n = 3) | Cell viability |
|---|---|---|
| HepG2 | Badge (Normal) | 100.0 ± 7.8 |
| HepG2 | Negative Control (Vector NK92) | 83.8 ± 2.1 |
| HepG2 | IL-7 × CCL19 NK92 cells | 33.2 ± 3.3 |
| HepG2 | CCL19 × IL-7 NK92 cells | 52.2 ± 3.5 |

TABLE 5
Secretion of Grz-B, IFN-γ and TNF-α

| Cancer cell | Experimental group | Grz-B | IFN-γ | TNF-α |
|---|---|---|---|---|
| lines | (n = 2) | (pg/mL) | (pg/mL) | (pg/mL) |
| HepG2 | Badge (Normal) | 592 ± 179 | N.D | N.D |
| HepG2 | Negative control | 1,259 ± 81 | 8 ± 1 | N.D |
| | (covector NK92) | | | |
| HepG2 | IL-7 × CCL19 | 2,573 ± 450 | 320 ± 28 | 195 ± 37 |
| | NK92 cells | | | |
| HepG2 | CCL19 × IL-7 | 1,460 ± 466 | 71 ± 3 | 33 ± 10 |
| | NK92 cells | | | |

*N.D: Not detected

As shown in Table 4, significantly higher HepG2 killing effect was observed in the experimental groups containing IL-7×CCL19 NK92 and CCL19×IL-7 NK92 cell lines compared to the negative control NK92 cell line. As shown in Table 5, factors related to HepG2 cell line killing effect were also secreted in higher amounts in the IL-7×CCL19 NK92 and CCL19×IL-7 NK92 cell lines compared to the negative control. Thus, both IL-7×CCL19 NK cells and CCL19×IL-7 NK cell lines can be applied as efficient anti-cancer therapeutics.

### Example 8

### Cancer Cell Killing Effect of 7×19 iNK Cells

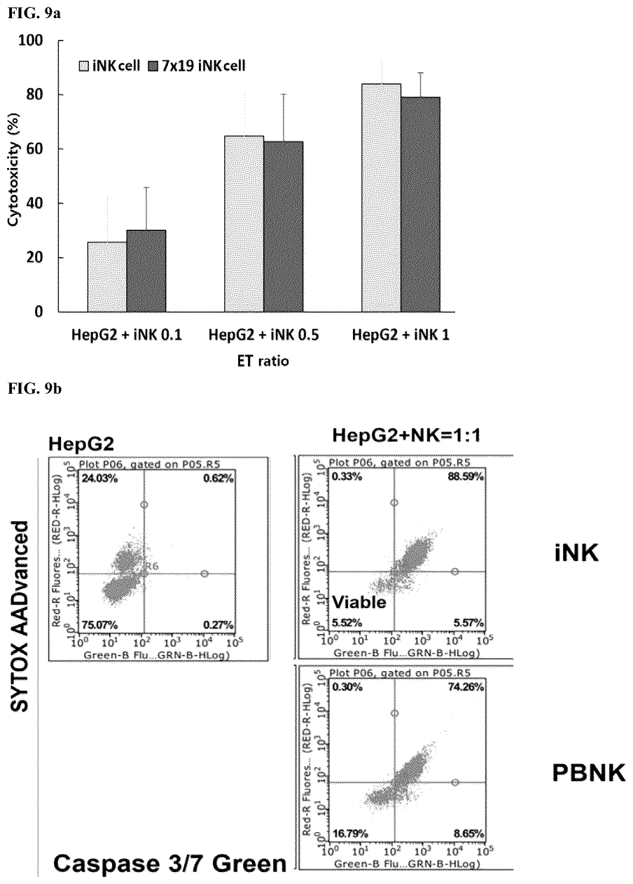

In vitro Comparison for the anti-cancer effects of iPS-derived NK cells with IL-7 and CCL19 genes introduced (7×19 iNK) and iNK cells without gene introduction indicated similar killing effects on the HepG2 cell line (FIG. 9a). This confirmed that the introduction of IL-7 and CCL19 genes did not cause a direct decrease in the killing ability of iNK cells against tumor cells. Therefore, it was expected that transgenic iNK cells (7×19 iNK) would exhibit a higher antitumor effect than non-transgenic iNK cells in an in vivo setting where an individual's immune system can be harnessed. To verify the killing ability of the transgenic iNK cells against tumor cells, the analysis for Sytox AADvanced (Dead cell) and caspase3/7 expression were performed to evaluate necrosis and apoptosis. As results, a significant killing effect on the HepG2 cell line was observed (FIG. 9b).

### Example 9

### T-Cell Chemotaxis Effect Induced by 7×19 iNK Cells

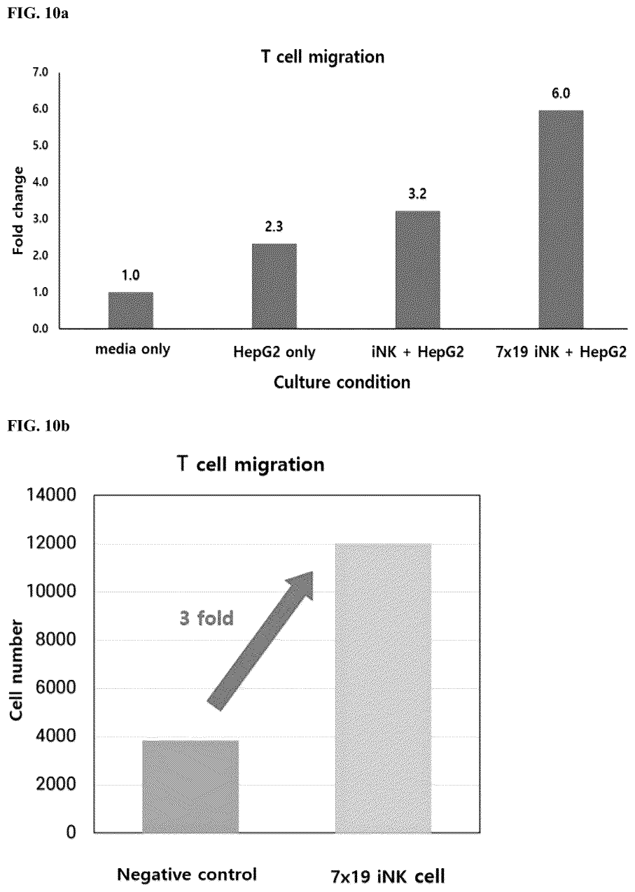

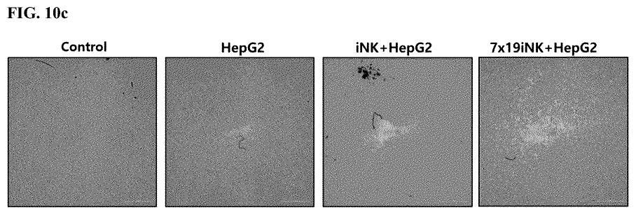

To determine the effect of IL-7 and CCL19 secreted by iPS-derived NK cells on T cell chemotaxis, T cells were seeded into the upper chamber of 12-well transwells (Corning, CAT #CLS3421) containing 5 μm polycarbonate filters, and the lower chamber was seeded with either no cells (media only) or cancer cells (HepG2); 7×19 iNK+cancer cells; and iNK cells without gene transduction+cancer cells, respectively. As results, no significant increase in T cell migration was observed when the lower chamber was seeded with cancer cells or cancer cells plus iNK cells without gene transfer compared to the control. however, co-culture of cancer cells with 7×19 iNK increased T cell migration in the upper chamber by approximately 2 to 3-fold. Furthermore, approximately 1.5 to 3-fold T cell migration was observed with 7×19 iNK compared to iNK cells without gene transfer (FIGS. 10a and 10b). To optically confirm T cell migration, the migrated cells were counted by unlabeled cell count using a Cytation5 cell imaging reader (10c, magnification×10). As results, induction of T cell migration by NK cells differentiated from induced pluripotent stem cells was significantly promoted by the introduction of IL-7 and CCL19 genes, indicating that the complementary immune responses of NK cells and the T cells recruited by them could achieve a multifaceted and synergistic tumor killing effect.

Having described specific embodiment of the present invention in detail above, it is to be understood that variants and modifications thereof falling within the spirit of the invention may become apparent to those skilled in this art, and the scope of this invention is to be determined by appended claims and their equivalents.

1. An immune cell that is differentiated from a pluripotent stem cell and expresses a nucleic acid molecule encoding IL-7 (interleukin-7) or a functional portion thereof; a nucleic acid molecule encoding CCL19 (C-C Motif Chemokine Ligand 19) or a functional portion thereof, or a combination thereof.

2. The immune cell according to claim 1, wherein the pluripotent stem cell expresses a nucleic acid molecule encoding IL-7 or a functional portion thereof; a nucleic acid molecule encoding CCL19 or a functional portion thereof; or a combination thereof.

3. The immune cell according to claim 1, wherein the pluripotent stem cell is at least one selected from the group consisting of an embryonic stem cell (ESC), an embryonic germ cell (EGC), an embryonic carcinoma cell (ECC) and an induced pluripotent stem cell (iPSC).

4. The immune cell according to claim 1, wherein the immune cell is an immune cell other than a T cell.

5. The immune cell according to claim 4, wherein the immune cell other than T cell is a natural killer cell.

6. A method for preventing or treating cancer or an infectious disease comprising administering to a subject in need thereof a composition comprising the immune cell according to claim 1 as an active ingredient.

7. A method for inducing proliferation or homing of a heterogeneous immune cell comprising administering to a subject in need thereof a composition comprising the immune cell according to claim 1 as an active ingredient.

8. The method according to claim 7, wherein the heterogeneous immune cell is a T cell or a dendritic cell.

9. A method for preparing a transformed immune cell comprising:
(a) introducing into a pluripotent stem cell a nucleic acid molecule encoding interleukin-7(IL-7) or a functional portion thereof; a nucleic acid molecule encoding C-C Motif Chemokine Ligand 19 (CCL19) or a functional portion thereof; or a combination thereof; and
(b) differentiating the cells obtained in the step (a) into an immune cell.

10. The method according to claim 9, wherein the immune cell is an immune cell other than a T cell.

11. The method according to claim 10, wherein the immune cell other than T cell is a natural killer cell.

12. A pluripotent stem cell expressing a nucleic acid molecule encoding IL-7 (interleukin-7) or a functional portion thereof; a nucleic acid molecule encoding CCL19 (C-C Motif Chemokine Ligand 19) or a functional portion thereof; or a combination thereof.

13. The pluripotent stem cell according to claim 12, wherein the pluripotent stem cell is at least one selected from the group consisting of an embryonic stem cell (ESC), an embryonic germ cell (EGC), an embryonic carcinoma cell (ECC) and an induced pluripotent stem cell (iPSC).


SEQUENCE LISTING (4 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 3 - DNA, 534 bp, Homo sapiens
  source 1..534: mol_type = genomic DNA
atgttccacgtctctttccggtacattttcgggctccccccactgatactggttcttctgccagtcgcatcatctgattgtgatatagagggtaaggacggaaaacaatatgaatctgtactgatggtgtcaatagaccagcttctcgatagcatgaaggaaattggtagtaattgtcttaataatgagttcaacttcttcaaacgccacatatgcgacgccaataaagagggtatgttcttgtttcgcgcggctaggaaactcagacaatttctcaaaatgaactctactggtgacttcgacctccaccttctgaaagtttccgaaggaactaccattcttctgaattgtacaggtcaggtcaaagggcgcaaaccagctgctcttggagaggcccagccaacgaagtcacttgaagaaaataagtctttgaaggaacaaaagaagctgaatgacctgtgctttctcaaaaggctgttgcaggagattaagacctgttggaacaaaatactcatggggaccaaggagcactga

SEQ ID NO: 4 - DNA, 297 bp, Homo sapiens
  source 1..297: mol_type = genomic DNA
atggcactgctcctcgccctgagtctcctggtactgtggacatcccctgcgcctacgctctcaggtacaaatgacgccgaagattgctgtctcagtgtaacccagaagcccataccgggttacattgtacggaacttccattatcttctgataaaagacggttgccgagtgcctgccgtggttttcacgacgttgagaggcagacagctgtgcgccccacccgaccagccgtgggtcgagcggatcatacaacgcttgcaaagaacgtccgcgaaaatgaaaaggcgctctagctaa